(19) United States
(12) Patent Application Publication
(10) Pub. No.: US 2016/0144159 A1
(43) Pub. Date: May 26, 2016

(54) CARBON DIOXIDE ADMINISTERING APPARATUS

(71) Applicant: NEOCHEMIR INC., Kobe-shi, Hyogo (JP)

(72) Inventor: Masaya TANAKA, Kobe-shi, Hyogo (JP)

(73) Assignee: NEOCHEMIR INC., Kobe-shi, Hyogo (JP)

(21) Appl. No.: 14/899,450

(22) PCT Filed: Jun. 19, 2014
(86) PCT No.: PCT/JP2014/066247

§ 371 Date: Dec. 17, 2015

(30) Foreign Application Priority Data
Jul. 4, 2013 (JP) 2013-140885

(51) Int. Cl. A61M 37/00

(52) U.S. Cl. CPC A61M 37/00; A61M 2202/0225; A61M 2202/0468; A61G 15/10

(57) ABSTRACT

A carbon dioxide administering apparatus to administer carbon dioxide to a part of the human skin that has an almost sealed Space (210) in which legs can be placed, Solvent tank (8) that reserves vaporable Solvent (8) in which carbon dioxide dissolves at the bottom of Space (210), Heating part (22) that heats up Solvent (8) in Solvent tank (23) to vaporize, Carbon dioxide supplying part (24) that supplies carbon dioxide into Space (210) above Solvent tank (23), and Adhesion part (25), to which floating droplets produced from Solvent (8) in Solvent tank (23) adhere, placed in Space (210) above Solvent tank (23). Legs can be placed in Space (210) above Adhesion part (25).

### TECHNICAL FIELD

The present invention relates to a carbon dioxide administering apparatus that administers carbon dioxide to a part of the human skin.

### BACKGROUND ART

It has already been known that various medical and cosmetic symptoms are improved when carbon dioxide is taken into the human body by transdermal absorption (see Patent Document 1). The main mechanisms of action of the carbon dioxide in these cases are a vasodilating action and a tissue oxygen partial pressure increasing action (see Non-Patent Document 1). Therefore, the effects of the carbon dioxide can be predicted by the presence or absence and degree of the redness of the skin that reflects the vasodilating action.

On the other hand, some means are already known to administer carbon dioxide to the human body through the skin or the mucous membrane (see Patent Documents 2 to 17).

### PRIOR ART DOCUMENTS

### Patent Document

Patent Document 1: JP 2000-319187 A

Patent Document 2: JP 3154634

Patent Document 3: JP 62-286922 A

Patent Document 4: JP 2008-259813 A

Patent Document 5: WO 2002/080941

Patent Document 6: WO 2004/002393

Patent Document 7: JP 07-171189

Patent Document 8: JP 2010-29265 A

Patent Document 9: JP 2010-29267 A

Patent Document 10: JP 2007-252871 A

Patent Document 11: JP 2006-34613 A

Patent Document 12: JP 2006-263253 A

Patent Document 13: JP 2006-34614 A

Patent Document 14: JP 2009-11695 A

Patent Document 15: JP 2009-183625 A

Patent Document 16: JP 2009-183626 A

Patent Document 17: JP 2009-254726 A

### Non-Patent Document

Non-Patent Document 1: Toshiki HIYOSHI, Jinko tansansen yokuzai ni yoru jokuso chiryo ni tsuite (Regarding Treatment of Bedsore by Bath Agent of Artificial Carbonated Spring), Sogo Riha 17(8), 605-9, 1989

### SUMMARY OF THE INVENTION

### Problem to be Solved by the Invention

But the administration methods described above have the following problems.

(1) The target skin must be bared. So it is hard for the skin that is concealed by clothing, bandage or the like to absorb carbon dioxide transdermally.
(2) Clothing, bandage or the like gets wet if they are not removed.
(3) The skin gets wet when carbon dioxide is absorbed transdermally.
(4) Carbon dioxide absorption efficiency is limited.

The objective of the present invention is to provide with a carbon dioxide administering apparatus that can easily be applied even to the skin covered with clothing or the like without wetting the clothing or the like, and further can improve the efficiency of the absorption of carbon dioxide remarkably.

### Means for Solving the Problem

A carbon dioxide administering apparatus to administer carbon dioxide to a part of the human skin of the present invention is characterized by having an almost sealed space in which a part of the human body can be placed, a solvent tank that reserves vaporable solvent in which carbon dioxide dissolves in the space, a heating part that heats up the solvent in the solvent tank to vaporize, a carbon dioxide supplying part that supplies carbon dioxide into the space, and an adhesion part, to which floating droplets produced from the solvent in the solvent tank adhere, placed above the solvent tank wherein a part of the human body can be placed in the space above the adhesion part.

Further, the present invention may favorably adopt the following constitution.

(a) The adhesion part is a flat plate member that is placed to cover the opening of the solvent tank in the planar view.
(b) Further, the apparatus has a space heating part that heats up the space above the adhesion part.
(c) Further, the apparatus has a human body heating part that directly raises the temperature of a part of the human skin which is placed inside the space.
(d) The solvent is water.
(e) Acidic material is dissolved in the solvent.
(f) Further, the apparatus has a sitting part that is equipped to enable the user to put his/her legs from the top side of the apparatus.

By the way, a part of the human body to which a carbon dioxide administering apparatus of the present is applied, is a concept containing all the parts of the human body to which the apparatus of the present invention can be applied without danger, and is not limited to the arm or the leg.

### Effects of the Invention

When a solvent is heated to vaporize, “floating droplets of the solvent” will also be produced together with “solvent vapor”. A “floating droplet of the solvent” means minute liquid of the solvent, which floats in the air, having a diameter of less than about 100 micrometer. Mist and steam are involved in it. That is, “floating droplets” wet a material to which they attach because they are liquid. On the other hand, “solvent vapor” means a gas state of the solvent molecules. “Gas state” is a state where solvent molecules are floating one by one and each molecule cannot be seen with the naked eye. That is, “vapor” does not wet a material to which it attaches because it is gas.

When carbon dioxide is supplied into the space where solvent is vaporized, solvent vapor or solvent molecule is thought to form “carbon dioxide-solvent complex” by reversible bonding with the supplied carbon dioxide molecule. Carbon dioxide dissolves into the floating droplets of the solvent.

The present invention can exert the following effects because carbon dioxide-solvent complex attaches the skin with little interference by floating droplets.

(i) The efficiency of the carbon dioxide transdermal absorption can be improved remarkably.
(ii) Wetting the clothing and the like and the skin can be prevented.
(iii) Carbon dioxide can easily be applied to the skin covered with clothing and the like.

### BRIEF DESCRIPTION OF THE DRAWINGS

FIG. 1 is a side-view rough sketch of the busy condition of Carbon dioxide administering apparatus of the present invention.

FIG. 2 is a front-view section plan of the Apparatus of FIG. 1.

FIG. 3 is a top-view oblique drawing of Machine of FIG. 1 whose Cylinder wall is opened.

FIG. 4 is a section plan showing a Solvent tank, Adhesion part, a Partition plate and a Footrest plate of the Apparatus of FIG. 1.

FIG. 5 is a front-view section plan of Apparatus of FIG. 1 having Space heating part and Human body heating part.

FIG. 6 is a front-view section plan of the simplified Apparatus of the Apparatus of FIG. 1 used in Example 4 and in Comparable Example 4.

### MODE FOR CARRYING OUT THE INVENTION

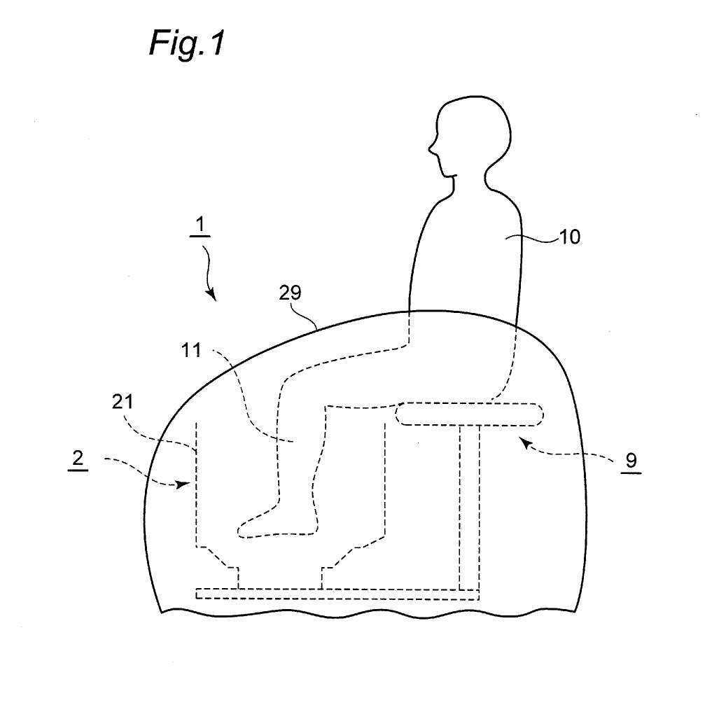

FIG. 1 is a side-view rough sketch of the busy condition of a Carbon dioxide administering apparatus of the present invention. Apparatus 1 is consisted of Main apparatus unit 2 and Sitting part 9. In Apparatus 1, User 10 sits on the Sitting part 9 and can place his/her Leg 11 in Main apparatus unit 2. The whole of Apparatus 1 can be covered with Sheet 29 including the lower half of the user's body.

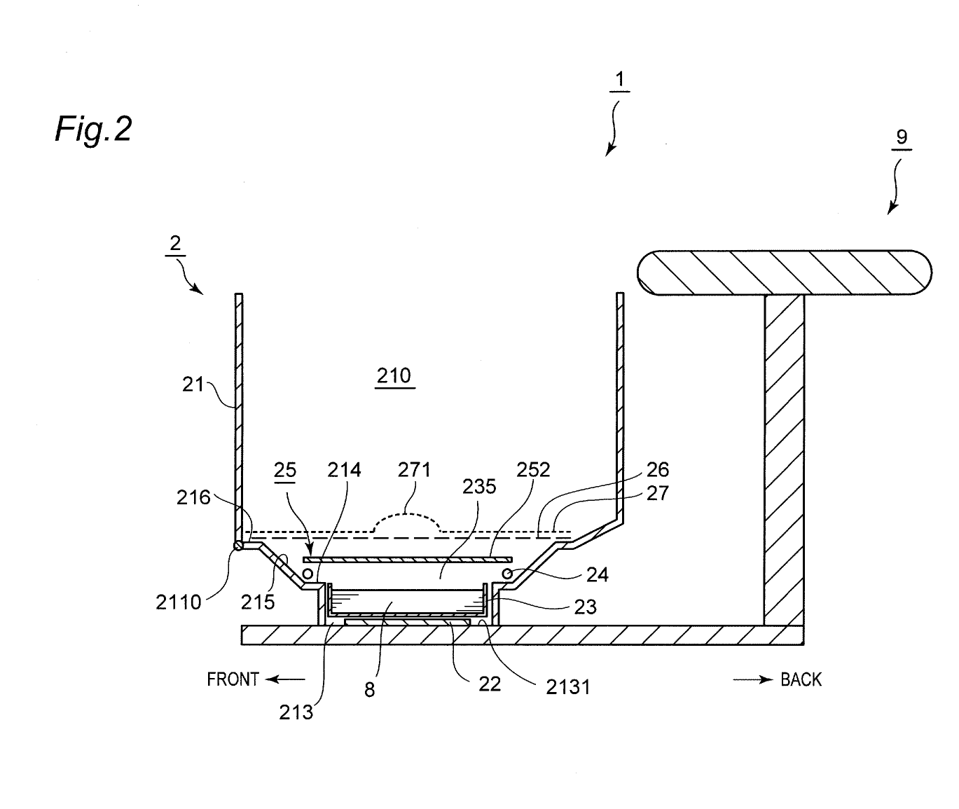

FIG. 2 is a front-view section plan of the Apparatus 1. Main apparatus unit 2 has Cylinder wall 21 with a bottom in which Leg 11 can be put from the top side of Cylinder wall 21. Space 210 can be almost sealed by covering Cylinder wall 21 with Sheet 21. Cylinder wall 21 can be opened by Front door 211 revolving forward on Hinge 2110.

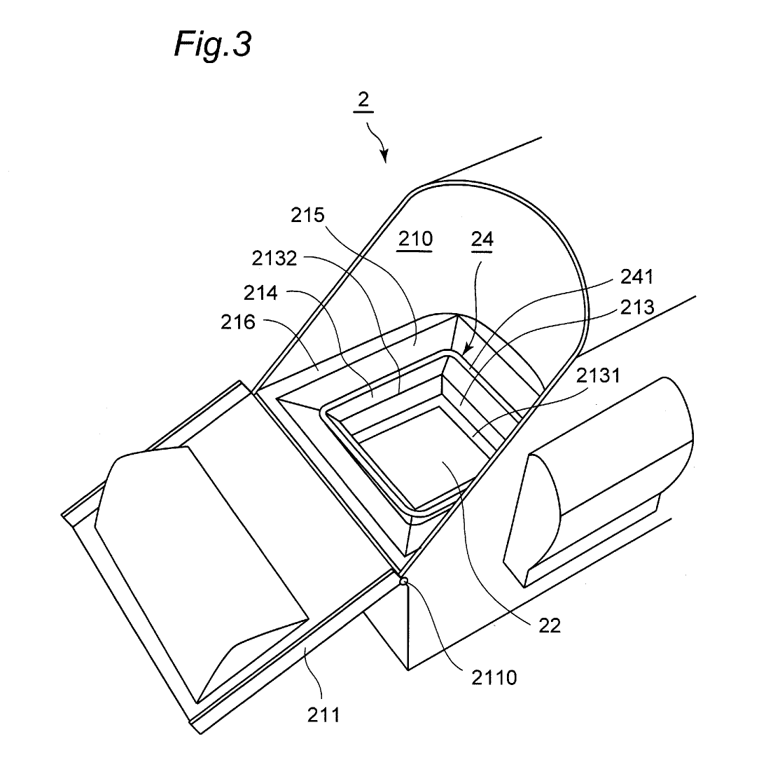

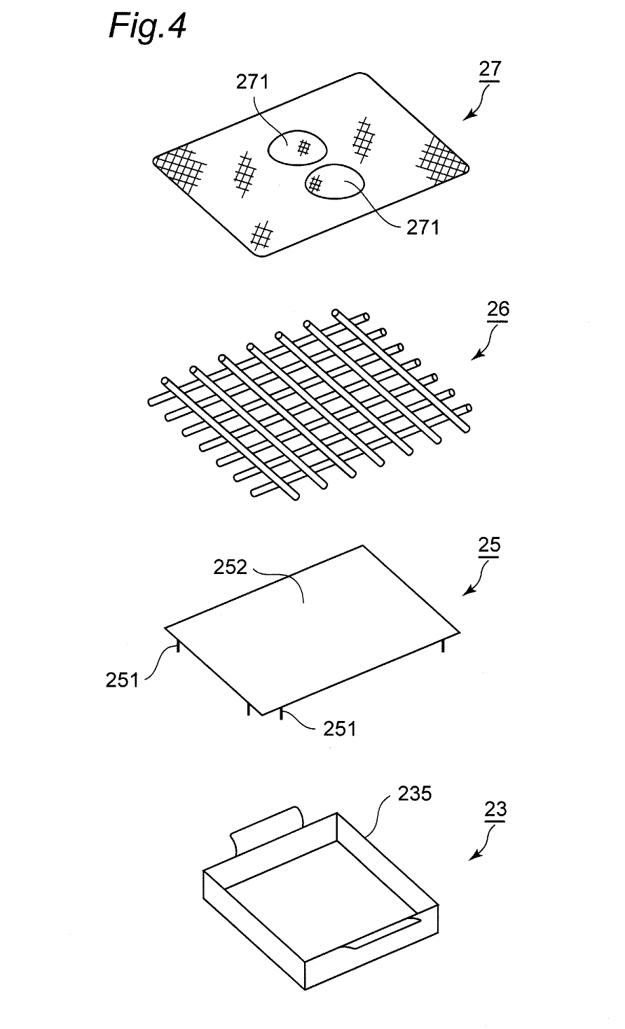

Cylinder wall 21 has Heating part 22, Solvent tank 23, Carbon dioxide supplying part 24, Adhesion part 25, Partition plate 26 and Footrest plate 27 from the lower position. FIG. 3 is a top-view oblique drawing of Main apparatus unit 2 whose Cylinder wall 21 is opened. FIG. 4 is a section plan showing Solvent tank 23, Adhesion part 25, Partition plate 26 and Footrest plate 27. FIG. 3 shows the state where the parts and units shown in FIG. 4 are not equipped yet.

Solvent tank 23 reserves Solvent 8. Solvent 8 is a medium that can vaporize and dissolve in carbon dioxide. Heating part 22 heats Solvent 8 in Solvent tank 23 to vaporize. Carbon dioxide supplying part 24 supplies carbon dioxide into Space 210 above Solvent tank 23. Adhesion part 25 is located in Space 210 above Solvent tank 23 to make floating droplets that are produced from Solvent 8 in Solvent tank 23 adhere. Partition plate 26 is located in Space 210 above Adhesion part 25 and Footrest plate 27 is located above Partition plate 26. So that Leg 11 can be placed in Space 210 above Footrest plate 27.

Further, concrete explanation is as follows.

Concavity 213, which has square shape in the planar view, is formed opening to the upper side at the bottom of Cylinder wall 21. Solvent tank 23 is a square-shaped container in the planar view embedded inside Concavity 213. A surface heater, which has square shape in the planar view, is equipped on Bottom 2131 of Concavity 213 to form Heating part 22. The heater is connected to the external power source (not illustrated) and heats the solvent tank attaching the bottom of Solvent tank 23.

First flat part 214 is formed around Opening 2132 of Concavity 213 in Cylinder wall 21. Carbon dioxide supplying part 24 has Pipe 241 which is parallel with and above First flat part 214. Pipe 241 has lots of carbon dioxide injection holes (not illustrated) in appropriate distances. The injection holes face the inside of Cylinder wall 21. Pipe 241 is connected to the carbon dioxide source (not illustrated) that is placed on the outside.

Adhesion part 25 is consisted of Flat member 252 that has Leg 251. Adhesion part 25 is placed to make Flat member 252 parallel to the surface of Solvent 8 in Solvent tank 23 when Leg 251 is placed on First flat part 214. Flat member 252 is a little bit wider than Opening 235 of Solvent tank 23 and covers Opening 235 in the planar view.

Cylinder wall 21 has Sloping part 215 adjacent to First flat part 214, and further Second sloping part 216. Partition plate 26 is consisted of square-shaped latticework plate in the planar view and has air conductivity. Partition plate 26 is placed inside Space 210 by placing its four sides on Second flat part 216. Footrest plate 27 is consisted of square-shaped mesh plate in the planar view and has air conductivity. Footrest plate 27 has two low Bumps 271 that are to be touched by the back of the sole of the foot.

Apparatus 1 of the above constitution is used as follows.

First, User 10 sits on Sitting part 9, puts the Legs 11 inside Cylinder wall 21 of Main apparatus unit 2 and then puts his/her foot on Footrest plate 27. On this occasion, it is favorable to put the sole on Bump 271. Then the whole of Apparatus 1 and the lower body of User 10 is covered by Sheet 29. Space 210 of Cylinder wall 21 is almost sealed by this procedure.

Then the operation switch (not illustrated) of Apparatus 1 is turned on. Heating part 22 heats Solvent 8 in Solvent tank 23 by this procedure. Solvent 8 is gradually vaporized by continuous heating. At this moment, Carbon dioxide supplying part 24 begins to work and carbon dioxide is supplied to the upper space of Solvent tank 23.

By the way, not only “vapor of Solvent 8” but also “floating droplets of Solvent 8” are produced at the same time when Solvent 8 is vaporized. A “floating droplet of the solvent” means minute liquid of the solvent, which floats in the air, having a diameter of less than about 100 micrometer in the present invention. On the other hand, “solvent vapor” means a gas state of the solvent molecules. “Gas state” is a state where solvent molecules are floating one by one and each molecule cannot be seen with the naked eye.

That is, vapor and floating droplets are produced by the vaporization of Solvent 8. And vapor or solvent molecules are thought to form “carbon dioxide-solvent complex” by reversible bonding with supplied carbon dioxide molecules. Further, the existence of this complex has been demonstrated by spectroscopic analysis (See “K. I. Peterson, W. Klemperer, J. Chem. Phys. 1984, 80, 2439” about a complex of carbon dioxide with water, for example). On the other hand, floating droplets adhere mainly to the back side of Flat member 252 of Adhesion part 25 and when they become large droplets, they drop down into Solvent tank 23. At this moment, the most of the produced floating droplets attach and adhere to Flat member 252 because it covers the whole Opening of 235 of Solvent tank 23 in the planar view. Accordingly, carbon dioxide-solvent complex goes up to Space 210 above Adhesion part 25, but the most of floating droplets do not go up. That is, carbon dioxide-solvent complex goes up further through Partition plate 26 and Footrest plate 27.

Accordingly, carbon dioxide-solvent complex attaches the skin of Leg 11, but floating droplets seldom attach the skin.

By the way, when carbon dioxide-solvent complex together with floating droplets attaches the skin, carbon dioxide that dissolved in floating droplets or “dissolved carbon dioxide” is absorbed because floating droplets adhere to the skin as if they cover it, but the carbon dioxide absorption from the carbon dioxide-solvent complex is interrupted. That is, “dissolved carbon dioxide” is the major carbon dioxide that is absorbed in this case. When water is solvent, for example, the concentration of dissolved carbon dioxide is only 0.1% under the atmospheric pressure. But the absorption of carbon dioxide from carbon dioxide-solvent complex is not interrupted because floating droplets merely attach the skin by Apparatus 1 of the above mentioned constitution. That is, the major of the absorbed carbon dioxide is the carbon dioxide from carbon dioxide-solvent complex. And the theoretical carbon dioxide content in the carbon dioxide-solvent complex is the maximum because carbon dioxide-solvent complex is a conjugate of solvent molecule and carbon dioxide molecule. Accordingly, the carbon dioxide absorption efficiency is remarkably improved by Apparatus 1 of the above mentioned constitution.

Moreover, carbon dioxide-solvent complex can attach the skin passing through clothing and the like because it is gas and it does not wet clothing and the like or the skin. Accordingly, Apparatus 1 of the above mentioned constitution can be used while the user wears clothing. Of course, Apparatus 1 of the above mentioned constitution may be used without wearing clothing.

As mentioned above, Apparatus 1 of the above mentioned constitution can be applied to the skin covered with clothing and the like. Moreover, clothing and the like or the skin does not get wet and further the carbon dioxide absorption efficiency can be remarkably improved. Accordingly, Apparatus 1 of the above mentioned constitution can exert the medical and cosmetic effects of transdermal/transmucosal absorption of carbon dioxide remarkably.

Further, any medium can be used as solvent without particular limitation while carbon dioxide can be dissolved in it and it is vaporizable. Of course, solvents fitting for use are liquid at normal temperature and innocuous. Specifically, water, organic solvent or inorganic solvent can be used. Tap water, distilled water, membrane filtered water, ion-exchanged water or electrolyzed water can be used as water without particular limitation. Monohydric alcohol such as methanol, ethanol, n-propanol, isopropanol, n-butyl alcohol, isobutyl alcohol, sec-butyl alcohol and ter-butyl alcohol; dihydric alcohol such as 1,3-butylen glycol, ethylene glycol, diethylene glycol and propylene glycol; glycol ether such as 2-phenoxyethanol can be used as organic solvent. Further, Water is the most favorable as solvent.

When water is used as solvent, the pH of water is not specifically limited but the pH less than 7 is preferred. Its reason is that carbon dioxide is consumed by the conversion into bicarbonate ion or carbonate ion in alkaline water, and this leads to less production of carbon dioxide-solvent complex. It is preferable to dissolve acidic compound in water to make its pH less than 7. Organic acid or inorganic acid can be used as acidic compound. Acidic electrolyzed water may also be used as water.

As carbon dioxide being supplied from Carbon dioxide supplying part 24, (a) the one produced from liquefied carbon dioxide, (b) the one produced from dry ice or (c) the one produced by the chemical reaction of carbonate and acid can be used, but it is not specifically limited to these.

Cylinder wall 21 should have heat tolerance property. It is preferable to choose component materials of Cylinder wall 21 properly according to the physical properties of the solvent. When acidic water is used as solvent, a material such as polypropylene which is acid resistant, for example, is preferably used.

As Flat member 252 of Adhesion part 25, (a) plate member, (b) fine-mesh cloth or (c) plate or cloth with fine through holes, for example, can be used. As component parts of these, (i) a metal or its alloy such as iron, copper, gold, silver or nickel, (ii) synthetic resin such as polyethylene, polypropylene, polyvinyl acetate, polystyrene, polycarbonate, polyacetal or polyamide can be used.

The higher concentration of carbon dioxide-solvent complex in Space 210 above Footrest plate 27 is preferable. More than 10% by volume of the concentration is preferable, more than 30% by volume is more preferable and more than 50% by volume is most preferable, for example.

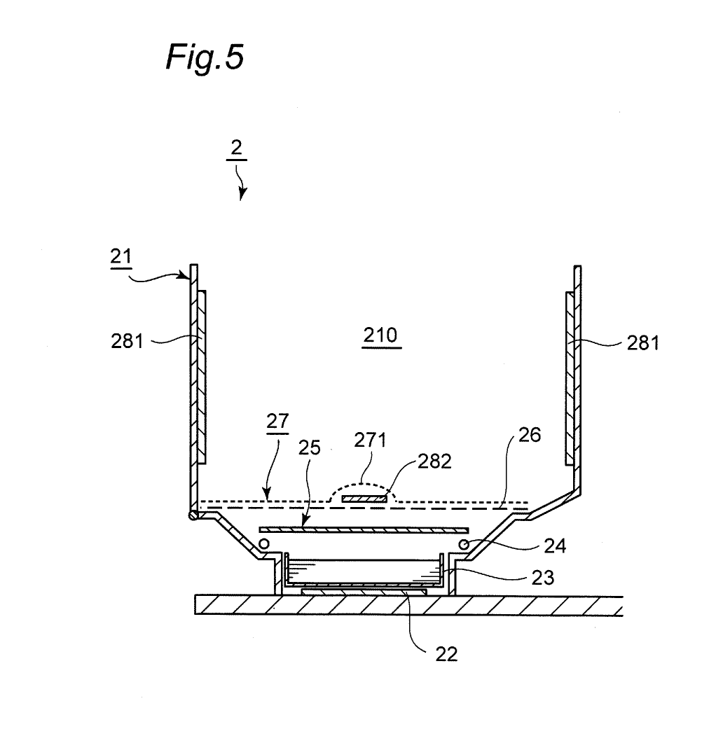

Further, Apparatus 1 of the above mentioned constitution preferably has a space-heating part that raises the temperature of Space 210 above Footrest plate 27. A space-heating part can be constituted by setting Heater 281 inside Cylinder wall 21 as shown in FIG. 5. Vapor volume of a solvent depends on the saturation vapor volume of the solvent at its temperature. The higher the temperature, the more the saturation vapor volume. Saturation vapor volume of water is 9.4 g/m³ at 10C.°, for example. On the other hand, saturation vapor volume of water is 30.4 g/m³ at 30C.°, which is more than three times. Accordingly, Apparatus 1 being set with a space-heating part can increase the number of solvent molecules that can bind to carbon dioxide by heating Space 210 above Footrest plate 27. This can lead to an increase in the medical and cosmetic effects of transdermal/transmucosal absorption of carbon dioxide by an increase in the production amount of carbon dioxide-solvent complex. Temperature in Space 210 above Footrest plate 27 is preferably over 20C.°, more preferably over 30C.° and further higher than the skin temperature is much more preferable, but it should be lower than the temperature at which the skin gets burned.

Apparatus 1 of the above mentioned constitution preferably has a human-body heating part that warms Leg 11 on Footrest plate 27 directly to raise the skin temperature (skin surface temperature). A human-body heating part can, for example, be constituted by setting Heater 282 inside Bump 271 of Footrest plate 27 as shown in FIG. 5. As Apparatus 1 which has a human-body heating part can raise the skin temperature to which carbon dioxide-solvent complex attaches, and not only absorption efficiency but also Bohr effect by carbon dioxide can be increased. In addition, the skin temperature is preferably 20 C.° and more preferably 30C.°, but it is preferable to avoid a high temperature, over 43C.° for example, that injures the tissue.

### EXAMPLES

### First Example

(Carbon Dioxide Administering Apparatus)

Apparatus 1 shown in FIG. 1 was used. Concrete sizes etc. are as follows.

Solvent tank 23 . . . 20 cm in length×20 cm in width×3 cm in depth

Flat plate member 25 . . . iron plate; 20 cm in length×20 cm in width×3 cm in thickness

Solvent 8 . . . 100 ml of water

(Usage Example)

(1) Operating Condition

In this apparatus 1, Solvent 8 was heated to 80C.° by Heating part 22 and 99.9% pure carbon dioxide was supplied to the upper space of Solvent tank 23 from the liquefied carbon dioxide cylinder using Carbon dioxide supplying part 24. In this case, no steam, or in other words, floating droplets were observed in Space 210 above Footrest plate 27. Humidity was 95% and carbon dioxide concentration was 50% by volume in Space 210.

(2) Working Condition

Users 10 were two males. Each of Users 10 put off the right one of the socks and exposed his right leg of Leg 11 by rolling up the hem of his pants while the left one of the socks and the hem of his pants were left as they were. Then each of Users 10 put his both legs of Leg 11 inside Cylinder wall 21 for 5 minutes.

(3) Evaluation Method

Skin redness induced by vasodilation was used for an indicator to evaluate the effect of carbon dioxide absorption. Three evaluators evaluated by the naked eye observation.

(4) Result

The right leg skin of the both of Users 10 turned red clearly and three evaluators judged strong vasodilation was induced. Further, the right leg of Leg 11 did not get wet completely. Three evaluators observed the left leg skin of the both of Users 10 when Users 10 put off the socks and rolled up the hem of their pants, and judged strong vasodilation was induced because the skin also turned red clearly. Further, both of the left one of the socks and their pants did not get wet completely.

### First Comparative Example

This is a comparative example against the First Example.

(Carbon Dioxide Administering Apparatus)

The only difference from the First Example is that the apparatus has no flat plate member.

### Usage Example

(1) Operating Condition

In this apparatus, Solvent 8 was heated to 80C.° by Heating part 22 and 99.9% pure carbon dioxide was supplied to the upper space of Solvent tank 23 from the liquefied carbon dioxide cylinder using Carbon dioxide supplying part 24. In this case, steam, or in other words, floating droplets was observed in Space 210 above Footrest plate 27. Humidity was 97% and carbon dioxide concentration was 42% by volume in Space 210. Further, lots of dew drops were formed inside Cylinder wall 21.

(2) Working Condition

It was the same as the First Example.

(3) Evaluation Method

It was the same as the First Example.

(4) Result

The right skin of the right legs of Legs 11 of Users 10 turned red, but three evaluators judged the extent of vasodilation was weaker than that of the First Example. Further, the right legs of Legs 11 were found got wet. Three evaluators observed the left leg skins of the both of Users 10 when Users 10 put off the socks and rolled up the hem of their pants, and judged no vasodilation was induced because the skin did not turn red. Further, both of the left one of the socks and their pants got wet.

### Second Example

(Carbon Dioxide Administering Apparatus)

The apparatus was the same as the First Example.

### Usage Example

(1) Operating condition

In this Apparatus 1, Solvent 8 was heated to 55C.° by Heating part 22 and 99.9% pure carbon dioxide was supplied to the upper space of Solvent tank 23 from the liquefied carbon dioxide cylinder using Carbon dioxide supplying part 24. In this case, no steam, or in other words, floating droplets was observed in Space 210 over Footrest plate 27. Humidity was 91%, carbon dioxide concentration was 90% by volume and the temperature was 36C.° in Space 210.

(2) Working Condition

User 10 was a male. User 10 put off the socks and exposed his legs of Leg 11 by rolling up the hem of his pants. Then User 10 put his both legs of Leg 11 inside Cylinder wall 21 for 5 minutes.

(3) Evaluation Method

The extent of sweating was observed by the naked eye observation. Further, User 10 put off both legs of Leg 11 from Cylinder wall 21 and blood flow of the insteps of Leg 11 was measured by laser-doppler flowmeter 1 minute after drying the Leg 11 with towel.

(4) Result

User 10 did not feel warm at first, but sweated after several tens of seconds and felt warm in his whole body and sweated on his paws after further several tens of seconds.

Blood flow increase after 1 minute was 38.0%.

### Second Comparative Example

This is a comparative example against the Second Example.

(Carbon Dioxide Administering Apparatus)

The apparatus was the same as the First Comparative Example.

### Usage Example

(1) Operating Condition

In this Apparatus 1, Solvent 8 was heated to 55C.° by Heating part 22 and 99.9% pure carbon dioxide was supplied to the upper space of Solvent tank 23 from the liquefied carbon dioxide cylinder using Carbon dioxide supplying part 24. In this case, steam, or in other words, floating droplets was observed in Space 210 above Footrest plate 27. Humidity was 91%, carbon dioxide concentration was 90% by volume and the temperature was 36C.° in Space 210.

(2) Working Condition

User 10 was a male. User 10 put off the socks and exposed his legs of Leg 11 by rolling up the hem of his pants. Then User 10 put his both legs of Leg 11 inside Cylinder wall 21 for 5 minutes.

(3) Evaluation Method

User 10 put off both legs of Leg 11 from Cylinder wall 21 and blood flow of the insteps of Leg 11 was measured by laser-doppler flowmeter 1 minute after drying Leg 11 with towel.

(4) Result

Leg 11 of User 10 got wet through from the beginning. Blood flow increase after 1 minute was 27.8% that was less than that of the Second Example.

### Third Example

(Carbon Dioxide Administering Apparatus)

The apparatus was the same as the First Example.

### Usage Example

(1) Operating Condition

In this Apparatus 1, Solvent 8 was heated to 55C.° by Heating part 22 and 99.9% pure carbon dioxide was supplied to the upper space of Solvent tank 23 from the liquefied carbon dioxide cylinder using Carbon dioxide supplying part 24. In this case, no steam, or in other words, floating droplets was observed in Space 210 above Footrest plate 27. Humidity was 91%, carbon dioxide concentration was 90% by volume and the temperature was 33C.° in Space 210.

(2) Working Condition

User 10 was a male. User 10 exposed his right arm and put it inside Cylinder wall 21 for 5 minutes.

(3) Evaluation Method

The extent of sweating was observed by the naked eye observation. Further, User 10 put off both legs of Leg 11 from Cylinder wall 21 and blood flow of the insteps of Leg 11 was measured by laser-doppler flowmeter 1 minute after drying Leg 11 with towel.

(4) Result

User 10 did not feel warm at first, but sweated after several tens of seconds and felt warm in his whole body and sweated on his hands and arms after further several tens of seconds.

Blood flow increase after 1 minute was 12.1%.

### Third Comparative Example

This is a comparative example against the Third Example.

(Carbon Dioxide Administering Apparatus)

The apparatus was the same as the First Example.

### Usage Example

(1) Operating Condition

In this Apparatus 1, Solvent 8 was heated to 55C.° by Heating part 22 and 99.9% pure carbon dioxide was supplied to the upper space of Solvent tank 23 from the liquefied carbon dioxide cylinder using Carbon dioxide supplying part 24. In this case, steam, or in other words, floating droplets was observed in Space 210 above Footrest plate 27. Humidity was 90%, carbon dioxide concentration was 90% by volume and the temperature was 41C.° in Space 210.

(2) Working Condition

User 10 was a male. User 10 exposed his left arm and put it inside Cylinder wall 21 for 5 minutes.

(3) Evaluation Method

User 10 put off his left arm from Cylinder wall 21 and dried it on a towel. Blood flow of the back of his left hand was measured by laser-doppler flowmeter 1 minute after drying the arm.

(4) Result

User 10 felt strong warm from the beginning. His left arm got sweat to the skin from the beginning.

Blood flow increase after 1 minute was 0.4%, which was much lower than that of Third Example.

### Fourth Example

(Carbon Dioxide Administering Apparatus)

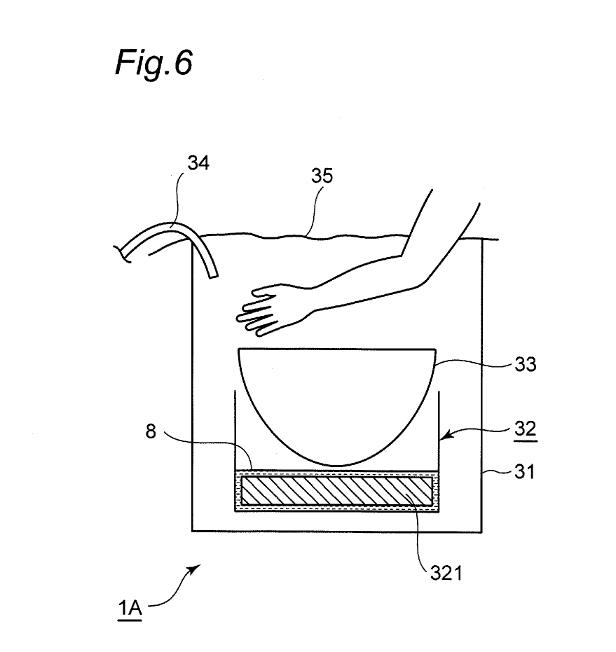

Carbon dioxide administering apparatus 1A shown in FIG. 6 was used. The Apparatus 1A is a simplified model of the Apparatus 1 shown in FIG. 1. Water bath 32 is installed in Container 31, water as solvent is stored in Water bath 32, Bowl 33 is placed above Water bath 32, and Tube 34 which is connected to the liquefied carbon dioxide gas cylinder is introduced inside Container 31. Water bath 32 has Heater 321 inside it. Container 31 is equivalent to Cylinder wall 21 of Apparatus 1. Water bath 32 is equivalent to Heating part 22 and Solvent tank 23 of Apparatus 1. Bowl 33 is equivalent to Adhesion part 25 of Apparatus 1. Tube 34 is equivalent to Carbon dioxide supplying part 24 of Apparatus 1.

Concrete constitution of Apparatus 1A shown in FIG. 6 is as follows.

Container 31 . . . plastic bucket with 35 cm in diameter×35 cm in height

Water bath 32 . . . 25 cm in diameter×15 cm in height, maximum output=700 W

Solvent 8 . . . 400 ml of water

Bowl 33 . . . 24 cm in diameter×15 cm in depth

### Usage Example

(1) Operating Condition

In this Apparatus 1, Container 31 was covered with Vinyl sheet 35 and the water temperature of Water bath 32 was set at 50 C.°. Carbon dioxide of 99.9% purity was introduced into Container 31 through Tube 34. Humidity inside Container 31 was 90%. The carbon dioxide concentration at the upper space of Container 31 was 80%.

(2) Working Condition

User 10 was a male. User 10 exposed his right arm and put it inside Container 31 for 3 minutes.

(3) Evaluation Method

The extent of sweating was observed by the naked eye. Further, User 10 put his right arm out from Container 31 and the blood flow of the back of his right hand was measured by laser-doppler flowmeter 1 and 5 minutes after the sweat was wiped with a towel.

(4) Result

User 10 felt no warm at first, but he felt warm in a minute in not only the right arm but also the shoulder, the back and other parts of the body. After he felt warm, his right arm got wet to the skin and his back and armpit were moist a little.

Blood flow increase after 1 minute was 17.8%.

Blood flow increase after 5 minutes was 6.7%.

### Fourth Comparative Example

This is a comparative example against the Fourth Example.

(Carbon Dioxide Administering Apparatus)

The only difference from Apparatus 1A of the Fourth Example is that the apparatus has not Bowl 33.

### Usage Example

(1) Operating Condition

This is the same as the Fourth Example.

(2) Working Condition

This is the same as the Fourth Example.

(3) Evaluation Method

This is the same as the Fourth Example.

(4) Result

The user felt intolerable hotness from the beginning and his right arm got wet soon.

Blood flow increase after 1 minute was 3.1%.

Blood flow increase after 5 minutes was 3.3%.

### INDUSTRIAL APPLICABILITY

Carbon dioxide administering apparatus of the present invention is highly useful in industry because it can increase the absorption efficiency of carbon dioxide remarkably.

### EXPLANATION OF REFERENCE NUMERALS

1, 1A: Carbon dioxide administering apparatus

210: Space

22: Heating part

23: Solvent tank

24: Carbon dioxide supplying part

25: Adhesion part

252: Flat plate member

281: Heater (space heating part)

282: Heater (human body heating part)

8: Solvent

9: Sitting part

1. A carbon dioxide administering apparatus to administer carbon dioxide to a part of the human skin, which is characterized by having;
an almost sealed space in which a part of the human body can be placed therein;
a solvent tank that reserves a vaporable solvent in which carbon dioxide dissolves placed in said space;
a heating part that heats said solvent in said solvent tank to vaporize;
a carbon dioxide supplying part that supplies carbon dioxide into said space;
an adhesion part, which adheres floating droplets that are produced from said solvent in said solvent tank, placed in said space above said solvent tank,
wherein a part of the human body can be placed in said space above said adhesion part.

2. The carbon dioxide administering apparatus according to claim 1, wherein said adhesion part is a flat plate member that is placed to cover the opening of said solvent tank in the planar view.

3. The carbon dioxide administering apparatus according to claim 1,
wherein said apparatus further has a heating part that raises the temperature inside said space above said adhesion part.

4. The carbon dioxide administering apparatus according to claim 1,
wherein said apparatus further has a human-body heating part to directly raise the skin temperature of a part of the human body that is placed in said space.

5. The carbon dioxide administering apparatus according to claim 1,
wherein said solvent is water.

6. The carbon dioxide administering apparatus according to claim 5,
wherein an acidic compound is dissolved in said solvent.

7. The carbon dioxide administering apparatus according to claim 1,
wherein said apparatus further has a sitting part and,
wherein said space is located in order to enable the person sitting on said sitting part to put his/her leg in the apparatus from the top side of the apparatus.

8. The carbon dioxide administering apparatus according to claim 2,
wherein said apparatus further has a heating part that raises the temperature inside said space above said adhesion part.

9. The carbon dioxide administering apparatus according to claim 2,
wherein said apparatus further has a human-body heating part to directly raise the skin temperature of a part of the human body that is placed in said space.

10. The carbon dioxide administering apparatus according to claim 3,
wherein said apparatus further has a human-body heating part to directly raise the skin temperature of a part of the human body that is placed in said space.

11. The carbon dioxide administering apparatus according to claim 8,
wherein said apparatus further has a human-body heating part to directly raise the skin temperature of a part of the human body that is placed in said space.

12. The carbon dioxide administering apparatus according to claim 2,
wherein said apparatus further has a sitting part and,
wherein said space is located in order to enable the person sitting on said sitting part to put his/her leg in the apparatus from the top side of the apparatus.

13. The carbon dioxide administering apparatus according to claim 3,
wherein said apparatus further has a sitting part and,
wherein said space is located in order to enable the person sitting on said sitting part to put his/her leg in the apparatus from the top side of the apparatus.

14. The carbon dioxide administering apparatus according to claim 4,
wherein said apparatus further has a sitting part and,
wherein said space is located in order to enable the person sitting on said sitting part to put his/her leg in the apparatus from the top side of the apparatus.

15. The carbon dioxide administering apparatus according to claim 8,
wherein said apparatus further has a sitting part and,
wherein said space is located in order to enable the person sitting on said sitting part to put his/her leg in the apparatus from the top side of the apparatus.

16. The carbon dioxide administering apparatus according to claim 9,
wherein said apparatus further has a sitting part and,
wherein said space is located in order to enable the person sitting on said sitting part to put his/her leg in the apparatus from the top side of the apparatus.

17. The carbon dioxide administering apparatus according to claim 10,
wherein said apparatus further has a sitting part and,
wherein said space is located in order to enable the person sitting on said sitting part to put his/her leg in the apparatus from the top side of the apparatus.

18. The carbon dioxide administering apparatus according to claim 11,
wherein said apparatus further has a sitting part and,
wherein said space is located in order to enable the person sitting on said sitting part to put his/her leg in the apparatus from the top side of the apparatus.
